(19) United States
(12) Patent Application Publication
(10) Pub. No.: US 2021/0161926 A1
(43) Pub. Date: Jun. 3, 2021

(54) Methods for Treatment of Brain Injury and Brain Dysfunction

(71) Applicant: Neuro BioFuels Limited, Colorado Springs, CO (US)

(72) Inventors: David J. STEPHENS, Colorado Springs, CO (US); Benjamin J. BURLINSON, Colorado Springs, CO (US)

(21) Appl. No.: 17/266,041

(22) PCT Filed: Aug. 7, 2019
(86) PCT No.: PCT/US2019/045440

§ 371 Date: Feb. 4, 2021

Related U.S. Application Data
(60) Provisional application No. 62/716,152, filed on Aug. 8, 2018.

(51) Int. Cl. A61K 31/7004; A61K 9/00; A61P 25/00

(52) U.S. Cl. CPC A61K 31/7004; A61P 25/00; A61K 9/0043; A61K 9/0053

(57) ABSTRACT

Methods are disclosed for treating brain injuries and brain dysfunction with the administration of dextrose. The disclosed methods of treatment with dextrose can be used to treat brain injuries such as concussions, conditions related to prior brain injuries such as chronic traumatic encephalopathy, and brain dysfunctions which may be caused by genetic or environmental factors, such as multiple sclerosis or post-traumatic stress disorder. The disclosed methods provide for safe and effective treatment of brain injuries and brain dysfunction in a cost-effective manner with minimal adverse side-effects.

### FIELD OF THE INVENTION

The present disclosure relates to the field of medical treatment of brain illnesses, diseases, disorders and injuries. In particular, the present disclosure relates to compositions and methods for treatment of brain illnesses, diseases, and disorders, including conditions such as multiple sclerosis and post-traumatic stress disorder. Further, the present disclosure relates to compositions and methods for treatment of brain injuries, such as concussions.

### BACKGROUND TO THE INVENTION

Brain injuries and brain dysfunction due to brain illness, diseases and disorders cause a great deal of suffering to affected patients, as well as their families and friends. Brain injuries commonly occur due to a swift impact to or sudden forceful motion of the head. Brain dysfunction due to brain illness, diseases and disorders can have many causes including prior brain injuries, genetic causes and environmental causes.

Treatment for brain injuries and brain dysfunction are wide ranging, but can be invasive, expensive, ineffective, and/or associated with adverse side effects. There remains a need in the art for treatments which are less invasive, financially accessible, effective and relatively free of side effects.

Brain injuries and brain dysfunction are associated with abnormal release of neurotransmitters as well as abnormal neuronal ion fluxes. More research is needed to fully understand the wide range of implications for brain biochemistry due to brain injuries and brain dysfunction. While much is not understood about neurometabolism, it is understood that the systems and processes involved in fueling the brain are highly complex and interdependent. Much like the peripheral nervous system, the brain has a great range of backstop measures to ensure that a consistent supply of fuel is always available.

The primary source of fuel for the brain is glucose (d), also known as dextrose. In recent studies, growing consensus in the scientific community indicates lactate as an additional substrate for brain metabolism. Ketones have also been widely studied as a source of neurobioenergetics.

Despite all the interest and investigation into the effects of brain injuries and brain dysfunction on neurometabolism, the identification, diagnosis, and treatment of brain injuries and brain dysfunction continues to be imprecise, inaccurate, and ineffective in many cases. Consequently, many brain injuries and brain dysfunctions are undetected, undiagnosed and/or inadequately treated.

Thus, there remains a need in the art for compositions and methods for effective treatment of traumatic brain injuries and brain dysfunction, including the symptoms associated with these conditions.

### SUMMARY OF THE INVENTION

Methods are disclosed for treating a patient experiencing a traumatic brain injury and/or brain dysfunction by administering to the patient a course of treatment comprising administration of at least one dose of dextrose.

In one aspect, a method is disclosed for treating traumatic brain injury, multiple sclerosis, or post-traumatic stress disorder comprising administering to a patient in need thereof a number of separate doses of dextrose in one day, wherein the number is selected from the group consisting of one, two, three, four, five and six separate doses of dextrose, further wherein each separate dose of dextrose comprises at least 10 grams of dextrose.

In another aspect, a method is disclosed for treating traumatic brain injury, multiple sclerosis, or post-traumatic stress disorder wherein the administration of dextrose is repeated over a course of treatment lasting at least 2 days, wherein the number of separate doses of dextrose administered on any one day of the course of treatment is selected independent of the number of separate doses of dextrose administered on any other day of the course of treatment and wherein the amount of dextrose comprised per separate dose is selected independent of the amount of dextrose comprised in any other separate dose administered during the course of treatment. In one aspect, over the course of treatment enough dextrose is administered to the patient to provide for cerebrospinal fluid levels of dextrose in the range of 45 to 80 mg/deciliter. In another aspect, the course of treatment lasts for N weeks, wherein N=1 to 52, further wherein N is not limited to a whole number.

In some embodiments, each dose of dextrose administered to a patient experiencing a brain injury or brain dysfunction comprises between 10 and 300 grams of dextrose per dose.

In some embodiments, each dose of dextrose administered to a patient experiencing a brain injury or brain dysfunction comprises between 20 and 30 grams of dextrose per dose.

In one aspect, the number of separate doses of dextrose administered to a patient experiencing a brain injury or brain dysfunction in one day is three or four.

In another aspect, the number of separate doses of dextrose administered to a patient experiencing a brain injury or brain dysfunction in one day is one or two.

In some embodiments, the separate doses of dextrose administered to a patient experiencing a brain injury or brain dysfunction comprise purified dextrose.

In some embodiments, each of the separate doses of dextrose administered to a patient experiencing a brain injury or brain dysfunction is administered orally. The orally administered dose can be in a dosage form selected from the group consisting of a liquid, a solid, a gel, a semisolid and a combination thereof. When the dosage form is a solid, the dosage form can be selected from the group consisting of a tablet, a capsule, a powder, a solid crystal and a combination thereof.

In some embodiments, each of the separate doses of dextrose administered to a patient experiencing a brain injury or brain dysfunction is administered intranasally. The intranasally administered dose can be in a dosage form selected from the group consisting of a liquid, a solid, a semisolid and a combination thereof.

In one aspect, the traumatic brain injury treated with dextrose is selected from the group consisting of concussion without loss of consciousness, concussion with loss of consciousness, post-concussion syndrome, mild traumatic brain injury, diffuse traumatic brain injury without loss of consciousness, diffuse traumatic brain injury with loss of consciousness, chronic traumatic encephalopathy, focal traumatic brain injury without loss of consciousness and focal traumatic brain injury with loss of consciousness. In a further aspect, the patient having the traumatic brain injury has been diagnosed as having experienced a concussion within 5 days of first administration of dextrose. In another aspect, the patient having the traumatic brain injury has been diagnosed as having experienced a concussion within 24 hours of first administration of dextrose.

In another aspect, the patient experiencing a brain injury or brain dysfunction

The following numbered paragraphs [0021]-[0040] contain statements of broad combinations of the inventive technical features herein disclosed:

1. A method for treating traumatic brain injury comprising administering to a patient in need thereof a number of separate doses of dextrose in one day, wherein the number is selected from the group consisting of one, two, three, four, five and six separate doses of dextrose, further wherein each separate dose of dextrose comprises at least 10 grams of dextrose.

2. The method of claim 1, wherein the administration of dextrose is repeated over a course of treatment lasting at least 2 days, wherein the number of separate doses of dextrose administered on any one day of the course of treatment is selected independent of the number of separate doses of dextrose administered on any other day of the course of treatment and wherein the amount of dextrose comprised per separate dose is selected independent of the amount of dextrose comprised in any other separate dose administered during the course of treatment.

3. The method of clam 1, wherein each dose of dextrose comprises between 10 and 300 grams of dextrose per dose.

4. The method of clam 1, wherein each dose of dextrose comprises between 20 and 30 grams of dextrose per dose.

5. The method of claim 1, wherein the number of separate doses of dextrose in one day is three or four.

6. The method of claim 1, wherein the number of separate doses of dextrose in one day is one or two.

7. The method of claim 1, wherein the separate doses of dextrose comprise purified dextrose.

8. The method of claim 1, wherein each of the separate doses of dextrose is administered orally.

9. The method of claim 8, wherein each of the separate doses of dextrose is in a dosage form selected from the group consisting of a liquid, a solid, a gel, a semisolid and a combination thereof.

10. The method of claim 9, wherein the dosage form is solid and selected from the group consisting of a tablet, a capsule, a powder, a solid crystal and a combination thereof.

11. The method of claim 2, wherein enough dextrose is administered to the patient to provide for cerebrospinal fluid levels of dextrose in the range of 45 to 80 mg/decilitre.

12. The method of claim 1, wherein each of the separate doses of dextrose is administered intranasally.

13. The method of claim 12, wherein each of the separate doses of dextrose is in a dosage form selected from the group consisting of a liquid, a solid, a semisolid and a combination thereof.

14. The method of claim 1, wherein the traumatic brain injury is selected from the group consisting of concussion without loss of consciousness, concussion with loss of consciousness, post-concussion syndrome, mild traumatic brain injury, diffuse traumatic brain injury without loss of consciousness, diffuse traumatic brain injury with loss of consciousness, chronic traumatic encephalopathy, focal traumatic brain injury without loss of consciousness and focal traumatic brain injury with loss of consciousness.

15. The method of claim 1, wherein the patient has been diagnosed as having experienced the traumatic brain injury within 5 days of first administration of dextrose.

16. The method of claim 1, wherein the patient has been diagnosed as having experienced a concussion within 5 days of first administration of dextrose.

17. The method of claim 1, wherein the patient has been diagnosed as having experienced a concussion within 24 hours of first administration of dextrose.

18. The method of claim 2, wherein the course of treatment lasts for N weeks, wherein N=1 to 52, further wherein N is not limited to a whole number.

19. A method for treating multiple sclerosis comprising administering to a patient in need thereof a number of separate doses of dextrose in one day, wherein the number is selected from the group consisting of one, two, three, four, five and six separate doses of dextrose, further wherein each separate dose of dextrose comprises at least 10 grams of dextrose.

20. A method for treating post-traumatic stress disorder comprising administering to a patient in need thereof a number of separate doses of dextrose in one day, wherein the number is selected from the group consisting of one, two, three, four, five and six separate doses of dextrose, further wherein each separate dose of dextrose comprises at least 10 grams of dextrose.

### DETAILED DESCRIPTION

Reference will now be made in detail to representative embodiments of the invention. While the invention will be described in conjunction with the enumerated embodiments, it will be understood that the invention is not intended to be limited to those embodiments. On the contrary, the invention is intended to cover all alternatives, modifications, and equivalents that may be included within the scope of the present invention as defined by the claims.

Unless defined otherwise, technical and scientific terms used herein have the same meaning as commonly understood by one of ordinary skill in the art(s) to which this invention belongs. Although any methods, devices, and materials similar or equivalent to those described herein can be used in the practice or testing of the invention, the preferred methods, devices and materials are now described.

All documents, or portions of documents, cited in this application, including but not limited to patents, patent applications, articles, books, and treatises, are hereby expressly incorporated by reference in their entirety.

As used herein, “a” or “an” may mean one or more than one of an item.

As used herein, “about” may mean up to and including plus or minus five percent, for example, about 100 may mean 95 and up to 105.

As used herein, “administer”, “administering” and “administration” are used interchangeably to include administration of dextrose by any route of administration. Providing a patient dextrose with instructions for self-administration is also considered administration of dextrose by the prescriber.

As used herein, “brain dysfunction” or “brain disorder” means any and all conditions categorized and coded/or in the Diagnostic and Statistical Manual of Mental Disorders, Fifth Edition (also referred to as the DSM-5).

As used herein, “course of treatment” means a period of continual treatment with dextrose, sometimes with variable dosage. The course of treatment may be considered to continue, even if dextrose is not administered on one or more days, as long as dextrose is subsequently administered within a week.

As used herein, “dextrose” means the D-isomer of glucose. Dextrose and glucose (d) are used interchangeably herein. Dextrose may be administered in a purified, substantially purified or unpurified form.

As used herein, “dose” or “dosage” means the amount of dextrose to be given.

As used herein, “route of administration” means the path by which the dextrose is taken into the body. Routes of administration include, but are not limited to, oral administration, intravenous delivery, and intranasal administration.

As used herein, “traumatic brain injury” or “brain injury” means any physical injury to the brain, including those resulting from one or more episodes of acute force to the head. The terms “traumatic brain injury” or “brain injury” include concussions. Concussion can include loss of consciousness or no loss of consciousness. The terms “traumatic brain injury” or “brain injury” include but are not limited to conditions which may occur later in time than the experience of force to the head, such as post-concussion syndrome and chronic traumatic encephalopathy.

The present disclosure describes the use of dextrose as a treatment for concussions, other traumatic brain injuries, and a wide range of brain disorders, illnesses, and mental illness. Further, dextrose treats many of the most troubling and persistent symptoms of concussions, traumatic brain injuries, and other brain disorders/mental illnesses.

Dextrose is commercially available without a prescription and is sold in a variety of dosage forms. Routes of administration of dextrose include, but are not limited to, oral administration, intravenous delivery, and intranasal administration. Oral dosage forms include, but are not limited to, dextrose tablets, dextrose gel, dextrose drinks, as well as bulk dextrose powdered products and dextrose beverages.

Dextrose is a naturally occurring monosaccharide. Naturally occurring monosaccharides include not only dextrose but also fructose, galactose, mannose, ribose and deoxyribose to name just a few. Dextrose is catabolized from disaccharides and polysaccharides through hydrolysis and can be obtained in limited amounts via more complex nutrition in a normal diet. However, dietary sources of dextrose are inadequate to provide the levels of dextrose necessary for the methods of treatment of the present disclosure.

It has been found through clinical study that there is a Dextrose Limiting Factor (DLF) and/or a Dextrose Regulating Factor (DRF) that emerges following concussive and traumatic brain injuries. This finding is supported by neuropsychological, vestibular, balance, and clinical report data that substantiate deficits in memory, visual-spatial, balance, visual, language, attentional, physical, emotional and psychological functioning following concussive and traumatic brain injuries, along with rapid elimination of concussive symptoms (headache, tinnitus, visual problems, pressure in the head and nasal cavities, anxiety, depression, anger, light sensitivity, etc.) following administration of doses of dextrose.

The dose of dextrose administered may be a little as about 10 grams per dose or as much as about 300 grams per dose, including all dosage amounts between 10 grams and 300 grams. Thus, each individual dosage of dextrose can be, while not limited to, about 10 grams, 15 grams, 20 grams, 25 grams, 30 grams, 35 grams, 40 grams, 45 grams, 50 grams, 55 grams, 60 grams, 70 grams 80 grams, 90 grams, 100 grams, 150 grams, 200 grams, 250 grams and 300 grams of dextrose.

The number of separate doses of dextrose administered in one day to a patient in need thereof can be one, two, three, four, five, six, seven, eight, nine or ten doses of dextrose.

The course of dextrose treatment can be one or more days. The course of dextrose treatment can last for N weeks, wherein N=1 to 52, wherein N is not limited to whole numbers. During the course of treatment, one or more days can pass without a patient receiving dextrose administration wherein the course or treatment is still considered to continue and include the subsequent administration of dextrose. The amount and frequency of dextrose administration can vary from day to day during the course of treatment.

While not wishing to be limited to any mechanism of action, it is believed that the brain injury or disorder may limit or regulate dextrose or dextrose uptake into cerebral tissue (e.g. cerebral spinal fluid, interstitial brain fluid and ventricular spaces), possibly causing neuronal hyperglycolyisis, which the administration of dextrose effectively treats.

A normal diet does not provide dextrose in sufficient amounts, or via the necessary pathways, to resolve the symptoms that are identified with concussive or traumatic brain injuries and brain disorders.

Individuals who have suffered concussive or traumatic brain injuries who are eating normal or even high-sugar diets continue to have symptoms related to concussions on neuropsychological assessment and balance, vestibular, and vision testing.

Individuals who have taken dextrose in tablet or liquid form, in addition to normal diet, have had statistically-significant improvement on follow-up neuropsychological testing.

For the DRF and/or DLF there is an inverted ratio of dextrose CSF and serum levels after injury. Current literature indicates a 0.6 ratio of CSF dextrose to serum in healthy individuals. This equates to CSF dextrose levels in the range of 45-80 mg/dL depending on peripheral serum levels. After neuronal damage during concussive force injuries the resulting cerebral hyperglycolyotic process reduces this ratio dramatically, to as low as 0.1-0.2.

Due to the efficiency of the body's insulin system, normal dietary intake of carbohydrates and complex sugars appears to be inadequate to drive serum blood sugar levels high enough to supply the brain with sufficient fuel after a lowered dextrose CSF/serum ratio. With an apparent limiting or regulation mechanism of cerebral dextrose, this ratio may be kept exceptionally low until adequate dextrose levels return to the brain and a resulting “reset” of normal dextrose uptake can be resumed. It is also important to note that the current medical knowledge base is highly limited when referring to dextrose transport to the brain. Careful consideration must be followed as undefined mechanisms may be present in dextrose transport within the brain and cerebral dextrose levels may be dramatically different between cerebral spinal fluid, brain interstitial fluid and any ventricular spaces. Types of dextrose transport between these areas are still yet to be discovered.

During head injury a significant cascade of neurochemical reactions takes place within the brain. During brain injury, whether impact or concussive inertial force, the brain, at a cellular level, undergoes a stretching and compression of neurons. This force “squeezes” neurotransmitters into the synaptic cleft and thus causes a systemic cascade of neuro reaction. This neuro reaction ultimately depolarizes the function of the brain.

The term commonly used to define a neuropathophysiological cascade resulting in depletion of sugar is hyperglycolysis, or the result of cellular energy demands in direct response to ionic fluxes. The result of hyperglycolysis is an arrested neuro-bioentergetic, fuelling system due to the exhaustion of available fuel.

The arrested neuro fuelling system creates long term covert and overt biological signs and symptoms due to a dynamic state of functional defueling within the brain. The brain elects to operate only the most important functions in any given time frame depending on external loads and stressors to compensate for the lack of adequate fuel to supply all energy demands. This accounts for most, if not all signs and symptoms of mTbi or concussion. This dynamic defueling is the bedrock of most brain conditions. As the brain automates function, defueled systems become the norm and are permanently arrested, causing a wide range of compensatory mal-adaptations that present as brain disease or dysfunction.

The brain fuels primarily on glucose (d), or more specifically dextrose. Glucose (d) is defined as a monosaccharide and is recognized in two forms, glucose (d) and glucose (L). As the body and brain cannot metabolize glucose (L), also known as cellulose, glucose (d) (i.e. dextrose) is understood to be a building block of neuro bioenergetics. Dextrose can be catabolized from more complex carbohydrates or sugars. This metabolic mechanism however is an expensive biological step and time consuming. With too great a latency period during this metabolism, pancreatic response produces an insulin reaction which shuttles most dextrose to the core or peripheral system where it is stored as glycogen rather than crossing the blood-brain barrier and fuelling the brain.

The clinical research underlying the present disclosure has found that oral administration of dextrose in appropriate therapeutic doses relieves all symptoms and signs of concussion. The administration of dextrose also has positive effects on a range of other brain injuries and brain dysfunctions as well.

Dextrose has been found to be useful in the treatment of a variety of brain injuries and dysfunctions, including but not limited to, multiple sclerosis, GluT1 deficiency syndrome, seizure control, chronic traumatic encephalopathy, vision and vestibular dysfunction, delayed and immediate recall mechanisms, attention, depression, anxiety, appetite control, sleep disturbances, and addictions.

In particular, dextrose is an effective treatment for mood disorders, including but not limited to, bipolar disorder type I, bipolar disorder type II, major depressive disorder, cyclothymia, dysthymia. Symptoms treated by dextrose for bipolar disorder types I and II and cyclothymia: manic symptoms, depressive symptoms, mood swings, anger, irritability frequency, intensity, and duration are all reduced by dextrose treatment as measured by performance on objective psychometric testing, clinician observation, and self and other report. Symptoms treated by dextrose for major depressive disorder and dysthymia: depressive symptoms, suicidal ideation, feelings of guilt, lack of energy, lack of motivation, appetite disruption frequency, intensity, and duration are all reduced by dextrose treatment as measured by performance on objective psychometric testing, clinician observation, and self-report.

Further, in particular, dextrose is an effective treatment for anxiety disorders, including but not limited to, generalized anxiety disorder, social anxiety disorder, separation anxiety disorder, panic disorder, and agoraphobia. Symptoms treated by dextrose for generalized anxiety disorder: anxiety, irritability, and avoidant behaviors have frequency, intensity, and duration all reduced by dextrose treatment as measured by performance on objective psychometric testing, clinician observation, and self and other report. Symptoms treated for social anxiety disorder, separation anxiety disorder, and agoraphobia: anxiety, irritability, avoidance of others, clinging to others, avoidance of public places have frequency, intensity, and duration are all reduced by dextrose treatment as measured by objective psychometric testing, clinician observation, and self and other report. Symptoms treated for panic disorder: panic attacks, avoidant behaviors, sleep difficulty have reduced frequency, intensity, and duration by dextrose treatment as measured by performance on objective psychometric testing, clinician observation, and self and other report.

In another aspect, dextrose is an effective treatment for seizure disorder/epilepsy. Symptoms treated by dextrose include seizure frequency, intensity, and duration which are reduced by dextrose treatment as measured by performance on objective psychometric testing, EEG results, clinician observation, and self and other report.

In another aspect, dextrose is an effective treatment for diabetes, including Type I, Type II and pre-diabetes. Symptoms treated by dextrose include elevated blood sugars, which are reduced by dextrose treatment as measured by blood glucometer, AIC tests, clinician observation, and self and other report.

In another aspect, dextrose is an effective treatment for dementias, including but not limited to, major neurocognitive disorder, minor neurocognitive disorder, Alzheimer's dementia, vascular dementia, dementia with Lewy bodies, and frontotemporal dementia. Symptoms treated by dextrose for all dementias included reduced frequency, intensity, and duration of memory difficulty, headaches, impulsivity, poor judgment, aphasias, ataxias, apraxias as measured by neuropsychological testing, clinician observation, and self and other report.

Dextrose is successful in treating these conditions because of its simplistic chemical form requiring no metabolic reaction to render it useful. Dextrose enters the glycolytic pathway immediately with no other anabolic or catabolic reaction being necessary. Outcome data indicates the introduction of dextrose provides a much needed energy supplement to brain metabolism, and helps “jump start” the neuro bioenergetic system back into primary fuelling mechanisms and away from automated secondary and tertiary fuelling systems. As full functional operation of the brain returns and compensatory mechanisms are reduced, signs and symptoms resolve.

The treatment of the above-listed conditions consists of the following steps: self-administration of dextrose 2-4 times per day and clinician monitoring and adjusting of the dose to ensure adequate dextrose levels are attained in the brain. For some of the conditions listed above, such as anxiety disorders, depressive disorders, concussive disorders, addictive disorders, psychotic disorders, etc., cognitive-behavioural therapy administered 2-4 times per month also promotes recovery from the conditions. Finally, visual, vestibular, attention, and memory drills are utilized in some cases to enhance recovery. The essential treatment for all conditions, however, is the dextrose treatment.

### EXAMPLES

The following examples are provided for illustrative purposes only and are not intended to limit the scope of the invention as defined by the appended claims. All examples described herein were carried out using standard techniques, which are well known and routine to those of skill in the art.

### Example 1

### Diagnosis: Chronic Traumatic Encephalopathy

68 year-old male. History of 20+ concussions and/or traumatic brain injuries. Neuropsychological test scores all at or below the 1^{st} percentile at time of first appointment. Headache, head pressure, balance, dizziness, anger, anxiety, depression all at 90^{th} percentile or higher at time of first appointment. Began with 20 grams of glucose (d) on initial appointment. This dose reduced all symptoms to below the 50^{th} percentile during initial appointment. Recommended increasing dose that day to 50 grams, 3 times per day. That dose reduced symptoms to 20^{th} percentile after 1 week. Increased dose to 100 grams 4 times per day. That dose eliminated all headaches, head pressure, with return of ability to “multi-task, no longer needing to hold on to the walls in the shower, and no more symptoms.” Neuropsychological test scores all at or above the 70^{th} percentile at conclusion of treatment. Headache, head pressure, balance, dizziness, anger, anxiety, and depression all resolved at conclusion of treatment. Glucose (d) discontinued.

### Example 2

Diagnosis: Post-Concussion Syndrome

17 year-old female. History of 3 identified concussions; most recent concussion 12 months prior to first appointment. Cumulative high school GPA: 4.3. Currently failing classes. Neuropsychological test scores ranged from 27^{th} percentile to 84^{th} percentile. Headache, tinnitus, light sensitivity, anxiety, depression ranged from 50^{th} to 90^{th} percentile at time of first appointment. Began with 12 grams of glucose (d) on initial appointment and all symptoms reduced to below the 20^{th} percentile by the end of the initial appointment. Did not take glucose (d) for the 3 days until next appointment. At that appointment anxiety in the 70^{th} percentile and depression in the 90^{th} percentile; headache, tinnitus, light sensitivity all above the 50^{th} percentile. Increased glucose (d) dose to 28 grams at time of appointment and all symptoms disappeared. Continued taking glucose (d) for 3 months at 28 grams 3 times per day. All symptoms continued to be resolved with no depression, anxiety, headache, tinnitus, or light sensitivity. Neuropsychological test scores ranged from 75^{th} percentile to 93^{rd} percentile at conclusion of treatment. Glucose (d) discontinued at conclusion of treatment with no return of symptoms.

### Example 3

Diagnosis: Concussion without Loss of Consciousness

17 year-old male. Treatment after concussion with a history of 1 prior concussion. Neuropsychological test scores ranged from 4^{th} percentile to the 41s^{t} percentile. Symptoms included headache, light sensitivity, tinnitus, difficulty paying attention at 80^{th} percentile at time of initial appointment. Began with 16 grams of glucose (d) at initial appointment that eliminated all symptoms by the end of the initial appointment. Recommended to take 16 grams 3 times per day until next appointment. At time of second appointment headache, head pressure, tinnitus, remained at 20^{th} to 30^{th} percentile. Glucose (d) dose recommended to increase to amount needed to eliminate all symptoms. Achieved elimination of symptoms at 80 grams of glucose (d) per dose, 4 times per day. Concluded treatment after 3 months and glucose (d) discontinued. All symptoms resolved. Neuropsychological test scores ranged from the 55^{th} to the 77^{th} percentile.

### Example 4

Diagnosis: Major Depressive Disorder, PTSD

64 year-old male. History of depressive episodes throughout his lifetime. Depression rated at 60^{th} percentile at time of initial appointment. History of rocket and gun attacks in Afghanistan during State Department missions. Anxiety, sleep disruption at 50^{th} percentile at time of initial appointment. Began with 20 grams of glucose (d) at initial appointment. Depression and anxiety reduced to 10^{th} percentile by the end of appointment. Recommended 40 grams of glucose (d) 4 times per day. Continued at that dose with no further symptoms of depression or anxiety for 4 months. Depression, anxiety, sleep problems eliminated at conclusion of treatment. Glucose (d) discontinued at that time.

### Example 5

Diagnosis: Post-Concussion Syndrome, Suspected Chronic Traumatic Encephalopathy

41 year-old male. History of “dozens of concussions.” Neuropsychological test scores ranged from the 1^{st} percentile to the 25^{th} percentile. Depression, anxiety, tinnitus, head pressure, memory problems, sleep difficulty, and anger all rated at the 80^{th} percentile or higher. Began with 40 grams of glucose (d) at initial appointment, which reduced all symptoms down to the 20^{th} percentile or lower by the conclusion of the initial appointment. Recommended dose of 100 grams of glucose (d) 4) times per day by the third appointment. This dose maintained symptoms at or below the 20^{th} percentile. Dose increased to 160 grams 4 times per day by the fourth appointment, which eliminated all symptoms. Treatment concluded after 5 months with no return of symptoms. Glucose (d) discontinued apart from occasional 40 to 50 gram one-time administration during times of high stress. Neuropsychological test scores ranged from 50^{th} to 78^{th} percentile at time of discharge from treatment.

### Example 6

Diagnosis: Concussion with LOC, Suspected Chronic Traumatic Encephalopathy

63 year-old female. Immediate reason for referral was motor vehicle accident with concussion with loss of consciousness. Neuropsychological test scores at the time of initial appointment were artificially elevated due to her familiarity with neuropsychological testing as a result of her career as a rehabilitation/occupational therapy specialist and having given neuropsychological screening instruments to many of her patients. Her neuropsychological test scores ranged from the 16^{th} percentile (despite specific familiarity with this task) to the 92^{nd} percentile. Depression, anxiety, headache, head pressure, mental “fogginess”, memory problems, “word-finding” difficulty, anger, balance problems all rated at the 80^{th} percentile or higher at the time of initial appointment. Began with 60 grams of glucose (d) at initial appointment, which reduced all symptoms to 30^{th} percentile or lower during initial appointment. Recommended dose of 100 grams 4 times per day at conclusion of initial appointment. At second appointment she reported that this dose reduced symptoms to the 30^{th} or 40^{th} percentile or lower. Recommended dose increase to 160 grams 4 times per day. At third appointment she reported this dose reduced symptoms consistently to 20^{th} percentile or lower, with some intermittent higher intensity of symptoms. By 5^{th} appointment she was taking between 200 and 240 grams 4 times per day, which eliminated all symptoms. She continued on this dose for 5 months, at which time all symptoms had been eliminated for a month and she discontinued use of glucose (d) apart from occasional doses of 20 to 40 grams during times of high stress. Neuropsychological test scores at the time of completion of treatment ranged from the 84^{th} to the 97^{th} percentile.

### Example 7

Diagnosis: Chronic Traumatic Encephalopathy

39 year-old male. History of “more than 50 fights with light flashes or dark spots in vision;” motor vehicle accident at 100-120 mph, which resulted in 30-day medically induced coma; incarceration in Department of Corrections for 3 years with multiple more fights resulting in concussion. Neuropsychological test scores at initial appointment ranged from 0.1 of the 1^{st} percentile to the 27^{th} percentile. Headache, head pressure, tinnitus, mental “fogginess,” anger, anxiety, depression, “word finding difficulty,” memory, dizziness, balance problems all rated at the 90^{th} percentile or higher at the initial appointment. Began with 160 grams of glucose (d), which eliminated all symptoms by the conclusion of the initial appointment. Has been taking 160-240 grams of glucose (d) 4 times per day, with no return of symptoms, for three weeks. Treatment is continuing.

### Example 8

Table 1 provides the results of dextrose treatment of patients with the following ages, genders, primary diagnoses, and comorbid diagnoses. Pre-RBANs Total refers to the pre-treatment patient score on the Repeatable Battery for the Assessment of Neuropsychological States (RBANS) as described in Duff, K., Hobson, V. L., Beglinger, L. J, O'Bryant, S. E. (2010). Diagnostic Accuracy of the RBANS in Mild cognitive impairment: Limitations on Assessing Milder Impairments. Archives in Clinical Neuropsychology, 25 (5), 429-441. Similarly, Post-RBANs Total refers to the post-treatment patient score for the same patient. The Hamilton score refers to the score on the Hamilton Anxiety Rating Scale as described in Hamilton M. The assessment of anxiety states by rating. Br J Med Psychol 1959; 32:50-55. The Beck score refers to the score on the Beck Depression Inventory. The majority of Hamilton Anxiety Rating Scale and Beck Depression Inventory scores were for assessment purposes at the beginning of treatment. For the treatment length specified, dextrose treatment was administered at a dosage range of 24 to 100 grams per dose and dosages of dextrose were administered 4 times per day for 7 days each week.

TABLE 1

| Length of | | | Primary | Comorbid | Pre-RBANS | Post-RBANS | Beck | Hamilton | |
|---|---|---|---|---|---|---|---|---|---|
| Treatment | AGE | Gender | Diagnosis | Diagnoses | TOTAL | TOTAL | Score | Score | Comments |
| 8 | 56.00 | F | Concussion | Chronic Post- | 90 | 119 | 5 | 11 | Post-Concussive |
| months | | | w/o LOC | concussion | | | | | Treatment: |
| | | | | Headache, | | | | | Significant increase |
| | | | | Sleep Disturbance | | | | | in Immediate/ |
| | | | | | | | | | Delayed Memory, |
| | | | | | | | | | Visuospatial/ |
| | | | | | | | | | Constructional, |
| | | | | | | | | | Language, and |
| | | | | | | | | | Attention abilities. |
| 10 | 25.00 | F | Concussion | Sleep Disturbance, | 80 | 80 | 20 | 27 | Post-Concussive |
| months | | | with LOC | Chronic Post- | | | | | Treatment: |
| | | | | concussive | | | | | Significant increase |
| | | | | Headache | | | | | in Visuospatial/ |
| | | | | | | | | | Constructional, |
| | | | | | | | | | Language and |
| | | | | | | | | | Delayed Memory |
| | | | | | | | | | Abilities. |
| 4 | 22.00 | M | Post- | Depression | 88 | 100 | N/A | N/A | Post-Concussive |
| months | | | Concussive | and Anxiety | | | | | Treatment: |
| | | | Syndrome | | | | | | Significant increase |
| | | | | | | | | | in Language abilities. |
| | | | | | | | | | Relational skills |
| | | | | | | | | | impaired by concussive |
| | | | | | | | | | injuries reported by |
| | | | | | | | | | client to have |
| | | | | | | | | | significantly improved. |
| 6 | 33.00 | F | Concussion | Chronic Post- | 60 | 86 | 7 | 0 | Post-Concussive |
| months | | | with LOC | concussion | | | | | Treatment: |
| | | | | Headache | | | | | Significant increase |
| | | | | | | | | | in Immediate/ |
| | | | | | | | | | Delayed Memory, |
| | | | | | | | | | Language, |
| | | | | | | | | | Visuospatial/ |
| | | | | | | | | | Constructional, |
| | | | | | | | | | Language, |
| | | | | | | | | | Attention abilities. |
| 8 | 57.00 | F | Concussion | Sleep | 61 | 92 | 24 | 20 | Post-Concussive |
| months | | | w/o LOC | Disturbance | | | | | & Bipolar DO |
| | | | | | | | | | Treatment: |
| | | | | | | | | | Significant increase |
| | | | | | | | | | in Immediate/ |
| | | | | | | | | | Delayed Memory, |
| | | | | | | | | | Visuospatial/ |
| | | | | | | | | | Constructional, |
| | | | | | | | | | Language, |
| | | | | | | | | | Attention abilities. |
| | | | | | | | | | Manic episodes |
| | | | | | | | | | decreased from |
| | | | | | | | | | 2x per month |
| | | | | | | | | | to none and |
| | | | | | | | | | psychotropic |
| | | | | | | | | | medications we |
| | | | | | | | | | discontinued by |
| | | | | | | | | | client's doctor at |
| | | | | | | | | | client's request. |
| 7 | 11.00 | M | Concussion | Chronic Post- | 102 | 108 | 4 | 7.5 | Post-Concussion |
| months | | | w/o LOC | concussion | | | | | Treatment: |
| | | | | Headache | | | | | Resolved issues |
| | | | | | | | | | of inattention |
| | | | | | | | | | and ADHD-like |
| | | | | | | | | | symptoms via a |
| | | | | | | | | | significant increase |
| | | | | | | | | | in Immediate/ |
| | | | | | | | | | Delayed Memory |
| | | | | | | | | | and Language |
| | | | | | | | | | abilities. |
| 7 | 13.00 | M | Adjustment | N/A | 81 | 88 | 3 | 3 | Resolved issues |
| months | | | Disorder | | | | | | with adjustment |
| | | | | | | | | | disorder via a |
| | | | | | | | | | significant increase |
| | | | | | | | | | in Immediate/ |
| | | | | | | | | | Delayed Memory |
| | | | | | | | | | and Visuospatial/ |
| | | | | | | | | | Constructional |
| | | | | | | | | | abilities. |
| 7 | 39.00 | F | Concussion | Depression | 91 | 105 | N/A | N/A | Post-Concussion |
| months | | | with LOC | and Anxiety | | | | | Treatment: |
| | | | | | | | | | resolved issues |
| | | | | | | | | | of depression/ |
| | | | | | | | | | anxiety and a |
| | | | | | | | | | significant increase |
| | | | | | | | | | in function of |
| | | | | | | | | | Visuospatial/ |
| | | | | | | | | | Constructional |
| | | | | | | | | | abilities, |
| | | | | | | | | | Language and |
| | | | | | | | | | Attention. |
| 5 | 59.00 | F | Concussion | Chronic Post- | 98 | 102 | 4 | 13 | Post-Concussion |
| months | | | w/o LOC | concussion | | | | | Treatment: |
| | | | | Headache | | | | | resolved severe |
| | | | | | | | | | depression and |
| | | | | | | | | | PTSD symptoms and |
| | | | | | | | | | a significant increase |
| | | | | | | | | | in function of |
| | | | | | | | | | Visuospatial/ |
| | | | | | | | | | Constructional abilities. |
| 6 | 57.00 | F | Concussion | Severe | 51 | 95 | 17 | 25 | Post-Concussion |
| months | | | w/o LOC | Depression | | | | | Treatment: |
| | | | | and Anxiety | | | | | Resolved depression/ |
| | | | | | | | | | anxiety and a |
| | | | | | | | | | significant reduction |
| | | | | | | | | | in anxiety/ |
| | | | | | | | | | depression via |
| | | | | | | | | | increase in |
| | | | | | | | | | functions of Memory |
| | | | | | | | | | (immediate/delayed), |
| | | | | | | | | | Visuospatial/ |
| | | | | | | | | | Constructional deficits, |
| | | | | | | | | | Language and |
| | | | | | | | | | Attention. |
| 7 | 60.00 | M | Concussion | Depression | 96 | 100 | 20 | 9 | Post-Concussion |
| months | | | w/o LOC | and | | | | | Treatment: |
| | | | | Inattention | | | | | Resolved depression/ |
| | | | | | | | | | anxiety and a |
| | | | | | | | | | significant increase |
| | | | | | | | | | in functions of Memory |
| | | | | | | | | | (immediate/delayed) |
| | | | | | | | | | and Visuospatial/ |
| | | | | | | | | | Constructional deficits |
| 5 | 51.00 | M | Concussion | Sleep | 78 | 89 | 18 | 12 | Post-Concussion |
| months | | | w/o LOC | Disturbance | | | | | Treatment: |
| | | | | | | | | | Resolved sleep |
| | | | | | | | | | disturbance, |
| | | | | | | | | | depression/anxiety |
| | | | | | | | | | and a significant |
| | | | | | | | | | increase in |
| | | | | | | | | | functions of Memory |
| | | | | | | | | | (immediate/delayed), |
| | | | | | | | | | Visuospatial/Constructional |
| | | | | | | | | | deficits, Object |
| | | | | | | | | | identification/ |
| | | | | | | | | | Semantic Fluency. |
| 5 | 15.00 | F | Concussion | Depression, | 92 | 106 | 5 | 6 | Post-Concussion |
| months | | | with LOC, | Inattention/ | | | | | Treatment: |
| | | | Chronic Post- | ADHD | | | | | Significant increase |
| | | | concussion | | | | | | in functions |
| | | | Headache | | | | | | of Memory |
| | | | | | | | | | (immediate/delayed), |
| | | | | | | | | | Language, |
| | | | | | | | | | Visuospatial/ |
| | | | | | | | | | Constructional |
| | | | | | | | | | deficits, Object |
| | | | | | | | | | identification/ |
| | | | | | | | | | Semantic Fluency. |
| 7 | 15.00 | F | Post- | Depression, | 87 | 108 | 18 | 22 | Post-Concussion |
| months | | | Concussive | Dyslexia, | | | | | Treatment: |
| | | | Syndrome, | Anxiety | | | | | Significant increase |
| | | | Chronic Post- | | | | | | in functions |
| | | | concussion | | | | | | of Memory |
| | | | Headache | | | | | | (immediate/delayed), |
| | | | | | | | | | Visuospatial/ |
| | | | | | | | | | Constructional |
| | | | | | | | | | deficits, Object |
| | | | | | | | | | identification/ |
| | | | | | | | | | Semantic Fluency/ |
| | | | | | | | | | Attention. |
| 9 | 30.00 | M | Concussion | Depression, | 54 | 92 | 21 | 30 | Resolved |
| months | | | with LOC | Anxiety, Panic | | | | | depression and |
| | | | | | | | | | anxiety (panic) |
| | | | | | | | | | via significant |
| | | | | | | | | | increase in |
| | | | | | | | | | functions of Memory |
| | | | | | | | | | (immediate/delayed), |
| | | | | | | | | | Visuospatial/ |
| | | | | | | | | | Constructional |
| | | | | | | | | | deficits, Object |
| | | | | | | | | | identification/ |
| | | | | | | | | | Semantic Fluency/ |
| | | | | | | | | | Attention. |
| 6 | 64.00 | F | Anxiety | Depression | 80 | 90 | 6 | 17 | Significant |
| months | | | | | | | | | reduction in |
| | | | | | | | | | anxiety/ |
| | | | | | | | | | depression via |
| | | | | | | | | | increase in |
| | | | | | | | | | functions of |
| | | | | | | | | | Memory |
| | | | | | | | | | (immediate/delayed), |
| | | | | | | | | | Visuospatial/ |
| | | | | | | | | | Constructional |
| | | | | | | | | | deficits, Object |
| | | | | | | | | | identification/ |
| | | | | | | | | | Semantic Fluency/ |
| | | | | | | | | | Attention. |
| 3 | 25.00 | M | Concussion | Sleep | 94 | 101 | 6 | 5 | Post-Concussion |
| months | | | w/o LOC | Disturbance | | | | | Treatment: |
| | | | | | | | | | Resolved sleep |
| | | | | | | | | | disturbance, |
| | | | | | | | | | increased functions |
| | | | | | | | | | of Memory |
| | | | | | | | | | (immediate) and |
| | | | | | | | | | Visuospatial/ |
| | | | | | | | | | Constructional abilities. |
| 7 | 57.00 | M | Concussion | Depression, | 108 | 111 | 16 | 27 | Post-Concussion |
| months | | | w/o LOC | Anxiety, Sleep | | | | | Treatment: |
| | | | | Disturbance | | | | | Resolved sleep disturbance, |
| | | | | | | | | | depression/ |
| | | | | | | | | | anxiety, tinnitus. |
| | | | | | | | | | Significantly |
| | | | | | | | | | increased functions |
| | | | | | | | | | of Memory |
| | | | | | | | | | (immediate/delayed), |
| | | | | | | | | | Attention and |
| | | | | | | | | | Visuospatial/ |
| | | | | | | | | | Constructional abilities. |
| 5 | 76.00 | F | Concussion | Diabetes | 63 | | 18 | 30 | Post-Concussion |
| months | | | with LOC | | | | | | Treatment: |
| | | | | | | | | | Increased functions |
| | | | | | | | | | of memory, |
| | | | | | | | | | eliminated headaches, |
| | | | | | | | | | sleep disturbance, |
| | | | | | | | | | increased irritability, |
| | | | | | | | | | depression and |
| | | | | | | | | | anxiety. |
| 5.5 | 18.00 | M | Concussion | N/A | 93 | 95 | 15 | 22 | Post-Concussion |
| months | | | w/o LOC | | | | | | Treatment: |
| | | | | | | | | | Increased functions |
| | | | | | | | | | of Visuospatial/ |
| | | | | | | | | | Constructional |
| | | | | | | | | | abilities and |
| | | | | | | | | | Language |
| | | | | | | | | | (Semantic Fluency/ |
| | | | | | | | | | Attention). |
| | | | | | | | | | Greatly improved |
| | | | | | | | | | symptoms of depression, |
| | | | | | | | | | anxiety, social |
| | | | | | | | | | avoidance and |
| | | | | | | | | | positive future |
| | | | | | | | | | orientation. |
| 5 | 34.00 | M | Autism | Post Chronic | 72 | 85 | 12 | 45 | Post-Concussion |
| months | | | | Concussion | | | | | Treatment: |
| | | | | | | | | | Significant improvement |
| | | | | | | | | | with Immediate/ |
| | | | | | | | | | Delayed Memory, |
| | | | | | | | | | Visuospatial/ |
| | | | | | | | | | Constructional, |
| | | | | | | | | | and Attention |
| | | | | | | | | | abilities. |
| 9 | 38.00 | M | PTSD and | Sleep | 63 | 79 | 26 | 38 | Post-Concussion |
| months | | | Depression | Disturbance | | | | | Treatment: |
| | | | | | | | | | Increased functions |
| | | | | | | | | | of Memory |
| | | | | | | | | | (immediate/delayed), |
| | | | | | | | | | Visuospatial/ |
| | | | | | | | | | Constructional |
| | | | | | | | | | deficits, Object |
| | | | | | | | | | identification/ |
| | | | | | | | | | Semantic Fluency/ |
| | | | | | | | | | Attention. |
| | 17.00 | M | Schizophrenia | Post | | | | | Eliminated psychotic |
| | | | | Concussive | | | | | symptoms, |
| | | | | Syndrome | | | | | increased |
| | | | | | | | | | socialization, |
| | | | | | | | | | stopped 4 meds |
| | 21.00 | F | Post | Type I | | | | | Insulin p blood |
| | | | Concussion | Diabetes | | | | | sugar lowered |
| | | | | | | | | | to low 100's |
| | | | | | | | | | (100-110) on a |
| | | | | | | | | | consistent basis. |
| | | | | | | | | | Less activation |
| | | | | | | | | | of insulin p |
| | 56.00 | M | Post | Type II | | | | | Blood Sugar |
| | | | Concussion | Diabetes and | | | | | reduced from |
| | | | | Chronic Pain | | | | | 250 to 120-130 |
| | | | | | | | | | consistently |
| | 15.00 | M | Concussion | Schizophrenia | | | | | Eliminated |
| | | | with LOC | | | | | | psychotic |
| | | | | | | | | | symptoms - |
| | | | | | | | | | discontinued |
| | | | | | | | | | antipsychotics |
| | 42.00 | M | Autism | Post | | | | | RBANS 2-4 SD |
| | | | | Concussive | | | | | increase from |
| | | | | Syndrome | | | | | pre to post testing; |
| | | | | | | | | | employed new; |
| | | | | | | | | | increased since |
| | | | | | | | | | interaction. |
| | | | | | | | | | Has not completed |
| | | | | | | | | | post RBANS |
| | | | | | | | | | significantly |
| | | | | | | | | | increased since |
| | | | | | | | | | interaction. |
| | 21.00 | M | Autism | Post | | | | | |
| | | | | Concussive | | | | | |
| | | | | Syndrome | | | | | |
| | 5.00 | M | Autism | | | | | | Reduced anxiety, |
| | | | | | | | | | eliminated |
| | | | | | | | | | classroom problems, |
| | | | | | | | | | eliminated outbursts |
| | 3.00 | M | Autism | | | | | | Improved speech, |
| | | | | | | | | | decreased anxiety, |
| | | | | | | | | | reduced emotional |
| | | | | | | | | | outbursts |
| | 19.00 | F | Autism | | | | | | Increased social |
| | | | | | | | | | interaction, |
| | | | | | | | | | reduced anxiety, |
| | | | | | | | | | improved speech |
| | 63.00 | M | Autism | | | | | | Increased |
| | | | | | | | | | socialization, |
| | | | | | | | | | improved memory, |
| | | | | | | | | | improved marriage |
| | 29.00 | M | Compulsive | | | | | | Eliminated use |
| | | | Sexual | | | | | | of pornography, |
| | | | Behavior, | | | | | | eliminated |
| | | | Childhood | | | | | | sexual acting |
| | | | Sexual Abuse, | | | | | | out |
| | | | PTSD | | | | | | |
| | 18.00 | F | Dissociative | | | | | | Eliminated |
| | | | Disorder, | | | | | | dissociative |
| | | | PTSD | | | | | | episodes, |
| | | | | | | | | | eliminated |
| | | | | | | | | | nightmares, |
| | | | | | | | | | eliminated |
| | | | | | | | | | PTSD symptoms |
| | 57.00 | M | Migraine | | | | | | Eliminated |
| | | | Headaches | | | | | | migraine |
| | | | | | | | | | headaches |
| | 38.00 | F | Migraine | | | | | | Eliminated |
| | | | Headaches | | | | | | migraine |
| | | | | | | | | | headaches |
| | 14.00 | M | Migraine | | | | | | Eliminated |
| | | | Headaches | | | | | | migraine |
| | | | | | | | | | headaches |
| | 56.00 | F | Migraine | | | | | | Eliminated |
| | | | Headaches | | | | | | migraine |
| | | | | | | | | | headaches |
| | 27.00 | M | Migraine | | | | | | Eliminated |
| | | | Headaches | | | | | | migraine |
| | | | | | | | | | headaches |

What is claimed is:

1. A method for treating traumatic brain injury comprising administering to a patient in need thereof a number of separate doses of dextrose in one day, wherein the number is selected from the group consisting of one, two, three, four, five and six separate doses of dextrose, further wherein each separate dose of dextrose comprises at least 10 grams of dextrose.

2. The method of claim 1, wherein the administration of dextrose is repeated over a course of treatment lasting at least 2 days, wherein the number of separate doses of dextrose administered on any one day of the course of treatment is selected independent of the number of separate doses of dextrose administered on any other day of the course of treatment and wherein the amount of dextrose comprised per separate dose is selected independent of the amount of dextrose comprised in any other separate dose administered during the course of treatment.

3. The method of clam 1, wherein each dose of dextrose comprises between 10 and 300 grams of dextrose per dose.

4. The method of clam 1, wherein each dose of dextrose comprises between 20 and 30 grams of dextrose per dose.

5. The method of claim 1, wherein the number of separate doses of dextrose in one day is three or four.

6. The method of claim 1, wherein the number of separate doses of dextrose in one day is one or two.

7. The method of claim 1, wherein the separate doses of dextrose comprise purified dextrose.

8. The method of claim 1, wherein each of the separate doses of dextrose is administered orally.

9. The method of claim 8, wherein each of the separate doses of dextrose is in a dosage form selected from the group consisting of a liquid, a solid, a semisolid and a combination thereof.

10. The method of claim 9, wherein the dosage form is solid and selected from the group consisting of a tablet, a capsule, a powder, a solid crystal and a combination thereof.

11. The method of claim 2, wherein enough dextrose is administered to the patient to provide for cerebrospinal fluid levels of dextrose in the range of 45 to 80 mg/decilitre.

12. The method of claim 1, wherein each of the separate doses of dextrose is administered intranasally.

13. The method of claim 12, wherein each of the separate doses of dextrose is in a dosage form selected from the group consisting of a liquid, a solid, a gel, a semisolid and a combination thereof.

14. The method of claim 1, wherein the traumatic brain injury is selected from the group consisting of concussion without loss of consciousness, concussion with loss of consciousness, post-concussion syndrome, mild traumatic brain injury, diffuse traumatic brain injury without loss of consciousness, diffuse traumatic brain injury with loss of consciousness, chronic traumatic encephalopathy, focal traumatic brain injury without loss of consciousness and focal traumatic brain injury with loss of consciousness.

15. The method of claim 1, wherein the patient has been diagnosed as having experienced the traumatic brain injury within 5 days of first administration of dextrose.

16. The method of claim 1, wherein the patient has been diagnosed as having experienced a concussion within 5 days of first administration of dextrose.

17. The method of claim 1, wherein the patient has been diagnosed as having experienced a concussion within 24 hours of first administration of dextrose.

18. The method of claim 2, wherein the course of treatment lasts for N weeks, wherein N=1 to 52, further wherein N is not limited to a whole number.

19. A method for treating multiple sclerosis comprising administering to a patient in need thereof a number of separate doses of dextrose in one day, wherein the number is selected from the group consisting of one, two, three, four, five and six separate doses of dextrose, further wherein each separate dose of dextrose comprises at least 10 grams of dextrose.

20. A method for treating post-traumatic stress disorder comprising administering to a patient in need thereof a number of separate doses of dextrose in one day, wherein the number is selected from the group consisting of one, two, three, four, five and six separate doses of dextrose, further wherein each separate dose of dextrose comprises at least 10 grams of dextrose.
